(19) United States
(12) Patent Application Publication
(10) Pub. No.: US 2022/0000138 A1
(43) Pub. Date: Jan. 6, 2022

(54) SUSPENSION OF POLYMERIZABLE MATERIALS IN A SOLID FAT MATRIX TO PREVENT AGGREGATION AND EXTEND SHELF LIFE OF FOOD MATERIALS

(71) Applicant: North Carolina State University, Raleigh, NC (US)

(72) Inventors: Gabriel Keith Harris, Raleigh, NC (US); Matthew Alan Robinson Sharp, Raleigh, NC (US)

(73) Assignee: North Carolina State University, Raleigh, NC (US)

(21) Appl. No.: 17/291,474

(22) PCT Filed: Nov. 11, 2019
(86) PCT No.: PCT/US2019/060714

§ 371 Date: May 5, 2021

Related U.S. Application Data
(60) Provisional application No. 62/758,310, filed on Nov. 9, 2018.

(51) Int. Cl. A23D 9/06; A23D 9/007; A23L 33/115; A23L 33/125; A23L 33/185; A23D 9/04

(52) U.S. Cl. CPC A23D 9/06; A23D 9/007; A23L 33/115; A23L 33/19; A23L 33/185; A23D 9/04; A23L 33/125

(57) ABSTRACT

Disclosed is a composition comprising a dispersion of an ingredient in a fat matrix, optionally wherein the ingredient is selected from the group comprising a macronutrient, a micronutrient, a dietary bioactive, a pharmaceutical ingredient, and combinations thereof, further optionally, wherein the dispersion is a uniform dispersion. In some embodiments, the macronutrient is a protein, a carbohydrate, a second type of fat in addition to the one forming the primary fat matrix, water, or a combination thereof. Also disclosed is a process for preparing a dispersion of an ingredient in a fat matrix, optionally wherein the ingredient is selected from the group comprising a macronutrient, a micronutrient, a dietary bioactive, a pharmaceutical ingredient, and combinations thereof, further optionally, wherein the dispersion is a uniform dispersion. In some embodiments, the process comprises: (a) heating the fat matrix to provide a melted or heat-plasticized fat matrix; (b) mixing the ingredient into the melted or heat-plasticized fat matrix, optionally wherein the ingredient comprises particles; and (c) cooling the mixture formed in step (b) to form the composition comprising a dispersion of an ingredient in a fat matrix, optionally wherein the ingredient is selected from the group comprising a macronutrient, a micronutrient, a dietary bioactive, a pharmaceutical ingredient, and combinations thereof, further optionally, wherein the dispersion is a uniform dispersion.

### CROSS-REFERENCE TO RELATED PATENT APPLICATIONS

This application claims benefit of U.S. Provisional Patent Application Ser. No. 62/758,310, filed Nov. 9, 2018. The disclosure of this U.S. Provisional Patent Application is incorporated herein by reference in its entirety.

### GRANT STATEMENT

This invention was made with government support under grant number SP4701-17-P-0049 awarded by the United States Department of Defense's Defense Logistics Agency (DOD/DLA). The government has certain rights in the invention.

### TECHNICAL FIELD

The presently disclosed subject matter relates in some embodiments to compositions comprising a dispersion of an ingredient, such as but not limited to a macronutrient, micronutrient, dietary bioactive, and/or pharmaceutical ingredient, in a fat matrix. The presently disclosed subject matter relates in some embodiments to a food product comprising a dispersion of a macronutrient in a fat matrix.

### BACKGROUND

Long chain macronutrients, such as starch and, in particular, protein, tend to self-associate and aggregate over time in food systems. In the case of protein in protein bars, this leads to bar hardening and the end of useful shelf life. The presently disclosed subject matter addresses these and other needs in the art.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

Provided in accordance with the presently disclosed subject matter is a composition comprising a dispersion of an ingredient in a fat matrix. In some embodiments, the ingredient is selected from the group comprising a macronutrient, a micronutrient, a dietary bioactive, a pharmaceutical ingredient, and combinations thereof, further optionally, wherein the dispersion is a uniform dispersion. In some embodiments, the macronutrient is a protein, a carbohydrate, a second type of fat in addition to the one forming the primary fat matrix, water, or a combination thereof.

In some embodiments, the fat matrix comprises a fat that is a solid at temperatures ranging up to 45° C. In some embodiments, the fat matrix comprises a fat that is semi-solid at temperatures ranging up to 40° C.

In some embodiments, the macronutrients are proteins and fats and the proteins and fats are present at ratio of between 1:1 and 1:3 protein to fat by weight. In some embodiments, the macronutrient is a protein of about 18 to 25 kiloDaltons. In some embodiments, the composition has a caloric value ranging from about 4 kilocalories per gram to about 8 kilocalories per gram.

In some embodiments, the ingredient comprises particles. In some embodiments, the ingredient particles have a particle size ranging from about 10 microns to about 200 microns. In some embodiments, the ingredient has a D90 value for particle size of about 100 microns or less.

In some embodiments, the protein comprises predominantly hydrophobic amino acid side chains, as determined by a technique selected from the group consisting of direct sequencing of amino acids, Wimley-White interface or octanol scales, water micro-droplet contact angle measurements, other common measures of peptide and protein hydrophobicity, or any combination of the foregoing. In some embodiments, the protein comprises a whey protein, a casein protein, an egg white protein, an insect protein, a plant protein, or combinations thereof. In some embodiments, the protein comprises at least about 20% of the caloric value of the composition, optionally at least about 25% of the caloric value of the composition.

In some embodiments, the carbohydrate comprises a sugar, a sugar syrup, a polyol, a flour, a starch, a fiber or hydrocolloid, or combinations thereof. In some embodiments, the composition further comprises an ingredient selected from the group consisting of a plasticizer, a thickener, an antioxidant, a chelator, a flavoring, and combinations thereof. In some embodiments, the composition has an average water activity below 0.85, optionally below 0.75.

In some embodiments, the fat is a fat selected from the group comprising cocoa butter, coconut oil, palm kernel oil, tristearin, and fractions and combinations thereof.

In some embodiments, the composition is provided in the form of a bar, a filling, a ganache, a pastry, a shortbread, an icing, a yogurt, a butter, a gel, a paste, a pellet, a sphere (with or without a food-grade candy shell), or an ovoid disc (with or without a food-grade chocolate, candy or gelatin shell/encapsulation).

Provided in accordance with the presently disclosed subject matter is a process for preparing a dispersion of an ingredient in a fat matrix. In some embodiments, the ingredient is selected from the group comprising a macronutrient, a micronutrient, a dietary bioactive, a pharmaceutical ingredient, and combinations thereof. In some embodiments, the dispersion is a uniform dispersion.

In some embodiments, the process comprises (a) heating the fat matrix to provide a melted or heat-plasticized fat matrix; (b) mixing the ingredient into the melted or heat-plasticized fat matrix; and (c) cooling the mixture formed in step (b) to form the composition comprising a dispersion of an ingredient in a fat matrix. In some embodiments, the ingredient comprises particles. In some embodiments, the ingredient is selected from the group comprising a macronutrient, a micronutrient, a dietary bioactive, a pharmaceutical ingredient, and combinations thereof. In some embodiments, the dispersion is a uniform dispersion.

In some embodiments, the fat matrix comprises a fat that is a solid at temperatures ranging up to 45° C. In some embodiments, the fat matrix comprises a fat that is semi-solid at temperatures ranging up to 40° C. In some embodiments, the macronutrients are proteins and fats and the proteins and fats are present at ratio of between 1:1 and 1:3 protein to fat by weight. In some embodiments, the macronutrient is a protein of about 18 to 25 kiloDaltons. In some embodiments, the composition has a caloric value ranging from about 4 kilocalories per gram to about 8 kilocalories per gram.

In some embodiments, the ingredient comprises particles. In some embodiments, the ingredient particles have a particle size ranging from about 10 microns to about 200 microns. In some embodiments, the ingredient has a D90 value for particle size of about 100 microns or less.

In some embodiments, the protein comprises predominantly hydrophobic amino acid side chains, as determined by a technique selected from the group consisting of direct sequencing of amino acids, Wimley-White interface or octanol scales, water micro-droplet contact angle measurements, other common measures of peptide and protein hydrophobicity, or any combination of the foregoing. In some embodiments, the protein comprises a whey protein, a casein protein, an egg white protein, an insect protein, a plant protein, or combinations thereof. In some embodiments, the protein comprises at least about 20% of the caloric value of the composition, optionally at least about 25% of the caloric value of the composition.

In some embodiments, the carbohydrate comprises a sugar, a sugar syrup, a polyol, a flour, a starch, a fiber or hydrocolloid, or combinations thereof. In some embodiments, the composition further comprises an ingredient selected from the group consisting of a plasticizer, a thickener, an antioxidant, a chelator, a flavoring, and combinations thereof. In some embodiments, the composition has an average water activity below 0.85, optionally below 0.75.

In some embodiments, the fat is a fat selected from the group comprising cocoa butter, coconut oil, palm kernel oil, tristearin, and fractions and combinations thereof.

In some embodiments, the composition is provided in the form of a bar, a filling, a ganache, a pastry, a shortbread, an icing, a yogurt, a butter, a gel, a paste, a pellet, a sphere (with or without a food-grade candy shell), or an ovoid disc (with or without a food-grade chocolate, candy or gelatin shell/encapsulation).

In some embodiments, the process comprises holding the melted or heat-plasticized fat matrix for a period of time prior to the mixing. In some embodiments, the holding of the fat matrix is carried out for 1 to 3 minutes. In some embodiments, the mixing the ingredient into the melted or heat-plasticized fat matrix using a low shear mixing method. In some embodiments, the method comprises forming the composition into a bar, a filling, a ganache, a pastry, a shortbread, an icing, a yogurt, a butter, a paste, a gel, a pellet, a sphere (with or without a food-grade chocolate, candy or gelatin shell/encapsulation), or an ovoid disc (with or without a food-grade chocolate, candy or gelatin shell/encapsulation). In some embodiments, forming the composition comprises forming the composition on a slab roller.

Thus, it is an object of the presently disclosed subject matter to provide a composition comprising a dispersion of an ingredient in a fat matrix, optionally wherein the ingredient is selected from the group comprising a macronutrient, a micronutrient, a dietary bioactive, a pharmaceutical ingredient, and combinations thereof, further optionally, wherein the dispersion is a uniform dispersion; and to provide a process for making the composition.

An object of the presently disclosed subject matter having been stated hereinabove, and which is achieved in whole or in part by the compositions and processes disclosed herein, other objects will become evident as the description proceeds when taken in connection with the accompanying Examples as best described herein below.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIG. 1 is a graph showing a comparison of fat separation from protein in 50/50 mixtures.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

In accordance with aspects of the presently disclosed subject matter, solid fats can be used to uniformly disperse protein particles, reducing the chance that this aggregation will occur and/or preventing this aggregation from occurring. In some embodiments of the presently disclosed subject matter, solid fats are used to disperse protein in general. In some embodiments of the presently disclosed subject matter, and more specifically, the type and particle size of the dispersed proteins, as well as the type and mixtures of solid fats, the processing required to uniformly disperse protein particles in a fat matrix, and the additional ingredients required to prevent fat migration and protein aggregation over the shelf life of a food.

Provided in accordance with some embodiments of the presently disclosed subject matter is a process that enhances the temperature stability and shelf life of complex, high energy density food systems by combining mixtures of particular fats with particular proteins, such as particular whey proteins. The homogenous distribution of protein in a solid fat matrix addresses the phenomena of protein polymerization in high-protein products, which leads to the hardening of food texture and limits shelf life. In some embodiments, the presently disclosed subject matter encompasses a unique 50/50 fat to protein (e.g., dairy protein) mixture that allows for enhanced shelf-stability, reduced protein (e.g., a dairy protein like a whey protein) isolate oxidation reactions, and low water activity. In a particular embodiment comprising this combination, it was shown to be stable at 55° C. for over six months in a high energy density protein bar model.

The presently disclosed formulations are adaptable to either sweet or savory products in a variety of forms such as bars, chocolates, and other confectionery products, translating into a new wave of protein-fortified products. The presently disclosed subject matter are adaptable into other products like candy bars, high-energy density or keto exercise products, or even icings or cake-like products. Some of the savory product flavors can include but are not limited to; bacon, chicken masala, cheese and crackers, beef burgundy, or any flavor desired to infuse into the solid matrix. Evidence of the absence of such a unique combination is readily apparent from the present market offerings.

In a representative embodiment of the presently disclosed formulation, whey protein isolates within a select particle size range (12 μm-130 μm), hydrophobicity, and a relatively low shear rate in either a vertical or horizontal mixer are employed. The formulation configured as a bar comprising 30 grams of protein per 100 grams product outperforms what is in the market currently in terms of shelf life and stability to high temperatures. Most other bar products currently on the market consisting of homogeneous mixtures are today are produced using extruder technology. The presently disclosed formulation when configured as a bar can be prepared using a temperature-controlled slab rolling method to distribute the product after low-shear homogenization. The use of fat as a carrier/enrober allows this matrix to prevent oxidation of the protein (e.g., whey protein) that generally occurs after 3 months of exposure to normal room conditions. Protein bars generally are not shelf-stable at high temperatures, 55° C., as most other products or their coatings start to become soft around 40° C. and melt at less than 50° C. In standard shelf life testing a product of the presently disclosed subject matter lasts for over six months at 55° C.; thus, predicting stability of three years at normal temperature conditions, using both standard and accelerated testing models. One reason for the very long shelf life of the resulting material is due to the low water activity of the presently disclosed formulation, which averages 0.58 Aw.

Thus, aspects of the presently disclosed subject matter include particular fat/protein ratios, such as 50/50 fat/protein ratios; particular combinations of specific fats and whey proteins; particular amino acid compositions in the proteins used; particular protein particle size ranges; the general superiority of whey proteins over mixed casein and whey; the ability of cocoa and palm kernel fats to work together effectively in this system; and the mixing and molding methods used for bar models. Overall, the aspects and qualities of the presently disclosed matrix and the process by which it is combined provides a novel, adaptable, and non-obvious option to improve the shelf life of high protein products.

### Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a component” includes a plurality of such components, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C and D.

As used herein, “dispersion” and “dispersed” refers to distribution of an ingredient, such as particles comprising an ingredient, within a fat matrix of a composition. “Homogeneously dispersed,” “evenly dispersed,” or “uniformly dispersed” refer to a distribution wherein sedimentation and/or agglomeration are minimized. For example, in some embodiments, “homogeneously dispersed,” “evenly dispersed,” or “uniformly dispersed”, is generally reflected by a lack of hardening of the composition and extended useful shelf life for the composition. In addition, the term “uniform dispersion” as used herein can refer to a homogenous dispersion of particles having a pre-determined concentration (weight/weight or weight/volume percent).

As used herein “sedimentation” refers to the settling of an ingredient, such as particles comprising an ingredient, typically in an aggregation or agglomeration. As used herein, in some embodiments, an agglomerate or an aggregate is a mass comprising particulate subunits formed via physical (hydrogen bonding, van der Waals, hydrophobic) or electrostatic forces. The resulting structure is called an “agglomerate.”

As used herein, “pharmaceutically acceptable” means that the material is suitable for administration to a subject (e.g., a human subject) and will allow desired treatment to be carried out without giving rise to unduly deleterious adverse effects. The severity of the disease and the necessity of the treatment are generally taken into account when determining whether any particular side effect is unduly deleterious.

As used herein, the term “effective” refers to provision of some improvement or benefit to the subject. Alternatively stated, an “effective amount” is an amount that will provide some alleviation, mitigation, or decrease in at least one clinical symptom in the subject. Those skilled in the art will appreciate that the effects need not be complete or curative, as long as some benefit is provided to the subject. The useful response can provide some alleviation, mitigation, or decrease in at least one clinical symptom in the subject. The terms also include an amount that will prevent or delay at least one clinical symptom in the subject and/or reduce and/or delay the severity of the onset of a clinical symptom in a subject relative to what would occur in the absence of the methods of the presently disclosed subject matter. Those skilled in the art will appreciate that the useful response need not be complete or curative or prevent permanently, as long as some benefit is provided to the subject.

As used herein, the terms “treatment,” “treat,” and “treating” refer to reversing, alleviating, delaying the onset of, inhibiting the progress of or preventing a disease or disorder. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example, to prevent or delay their recurrence.

As used herein, the term “low shear rate” refers to a shear rate of less than or equal to 100 rpm or (multiplying by an approximate 1.7 conversion factor) as less than or equal to 170 reciprocal seconds or seconds⁻¹. This definition is based on the optimal rpm values for mixers used to produce bar products. For a reference on rpm vs shear rate, please see Berk Z., Chapter 7, Mixing, In Food Process Engineering. 2013. Food process engineering and technology. Amsterdam, Academic Press, 2013.

### Compositions

An aspect of the presently disclosed subject matter is that by finely separating out protein particles in a fat medium, proteins are less likely to interact and form hard polymers. Bar hardening is what typically limits shelf life of protein bars. Another aspect of the presently disclosed subject allows for the creation of highly calorie dense foods that will deliver large amounts of calories in small packages. While the presently disclosed subject matter is primarily designed for proteins, it can also function to prevent the association of carbohydrates as well. Dispersing materials with some hydrophobic characteristics into fat can further stabilize the system, as can providing fine particle size dispersion of non-fat components (e.g., protein, sugar, vitamins, and minerals, etc.) in a fat matrix. In some embodiments, fine, uniform dispersion of protein, sugar, vitamin, and other particles in a fat (e.g., solid fat) matrix provides a high calorie, high nutrient density composition capable of withstanding temperatures of 40 degrees Celsius for extended periods of time without deformation or significant loss of flavor and nutritional properties over a two-year shelf-life (as estimated by accelerated storage testing).

In accordance with aspects of the presently disclosed subject matter, provided is a composition comprising an ingredient, such as but not limited to a macronutrient, micronutrient, dietary bioactive, and/or pharmaceutical ingredient, in a fat matrix. In some embodiments, the dispersion is a uniform dispersion. In some embodiments, the macronutrient is a protein, a carbohydrate, a second type of fat in addition to the one forming the primary fat matrix, water, or a combination thereof. In some embodiments, the composition comprises a dispersion of protein and other macronutrients or micronutrients into a solid fat matrix that maximizes calorie density, flavor, and shelf stability. In some embodiments, a food product composition comprising a bar is more calorie-dense than other bars currently available in the art. In some embodiments, the composition has a caloric value ranging from about 4 kilocalories per gram to about 8 kilocalories per gram. By way of elaboration and not limitation, 4 kilocalories per gram can be achieved through inclusion of a protein or a carbohydrate as the only macronutrient. Additionally, 8 kilocalories can be achieved as a weighted average of 1:3 protein to fat.

In accordance with aspects of the presently disclosed subject matter, approaches are provided for creating an anaerobic environment. An anaerobic environment has risks and benefits. A risk is Clostridium botulinum (C. bot) outgrowth, which can be avoided via water activity control as described herein below. A benefit is that nutrients, like fat, protein, and vitamins that can oxidize, will be prevented from doing so by a low or no oxygen environment. Thus, in some embodiments, approaches in accordance with the presently disclosed subject matter comprise dispersing nutrients in a fat matrix that does not result in the outgrowth of C. bot spores or other anaerobes of concern. The United States Food and Drug Administration (FDA) indicates that C. bot does not produce toxin at water activities below 0.85. In some embodiments, shown herein below in the Examples, a food product composition in accordance with the presently disclosed subject matter has an average water activity of 0.51 and 0.65 for a chocolate bar and a cheese bar, respectively. In some embodiments, a composition in accordance with the presently disclosed subject matter has an average water activity below 0.85, optionally below 0.75, further optionally below 0.70, further optionally below 0.65, further optionally below 0.60, further optionally below 0.55.

In some embodiments, certain micronutrients and/or dietary bioactive ingredients, for example, vitamins and minerals, are suspended in the solid fat matrix in order to prevent lipid oxidation and/or flavor changes over time. Given the high fat nature of the product, steps can be taken to prevent contact of transition metals, photosensitizing vitamins such as riboflavin, and other initiators and promoters of lipid oxidation. This is accomplished, for example, via the use of established nutrient encapsulation technologies.

In some embodiments, the presently disclosed subject matter provides a high calorie, high protein bars that will not harden and, therefore have extended shelf lives. In some embodiments, the bar has a caloric value ranging from about 4 kilocalories per gram to about 8 kilocalories per gram.

In some embodiments, the presently disclosed subject matter provides lower-calorie confections. To elaborate, fat has 9 kcal/g and protein has 4 kcal/g. This means that adding non-fat materials effectively dilutes the calorie content of high fat products.

In some embodiments, a composition of the presently disclosed subject matter comprises 25 to 35 grams protein per 100 grams of material, such as 25, 26, 27, 28, 29, 30, 31, 32, 33, 34, or 35 grams of protein per 100 grams material.

In some embodiments, the fat matrix comprises a fat that is a solid at temperature ranging up to 45° C., including solid at 40° C., 41° C., 42° C., 43° C., 44° C., and 45° C. In some embodiments, the fat matrix comprises a fat that is semi-solid at temperatures ranging up to 40° C., including semi-solid at 35° C., 36° C., 37° C., 38° C., 39° C., and 40° C. Cocoa butter, coconut oil, palm kernel oil, tristearin, and fractions and combinations thereof, are representative fats used to achieve a high-melting bar. Other lower melting fats, or semi-solid fats and fat mixtures (e.g., mono-unsaturated fats such as triolein mixed with cocoa butter, coconut oil, palm kernel oil, or tristearin), are employed for the creation of pastes. Fully hydrogenated, zero trans fats (e.g., fully hydrogenated soybean, coconut, or palm kernel oil) are also used to provide high-melting properties. In embodiments comprising chocolate, a chocolate liquor can be added, which provides additional fat content. In some embodiments, the matrix comprises a mixture of fats, such as a mixture of cocoa butter and palm kernel oil. In some embodiments, the mixture of cocoa butter and palm kernel oil comprises 10% by weight palm kernel oil. In some embodiments, palm kernel oil can range between 5% and 50% by weight (5/95 to 50/50 ratio) with cocoa butter, for example, 5, 10, 15, 20, 25, 30, 35, 40, 45, or 50% palm kernel oil.

The fat matrix is hydrophobic. Proteins can vary in hydrophobicity/hydrophilicity. In some embodiments using dairy protein (for example, whey protein), the protein comprises high aspartic acid (8.0-9.5 mg/100 g), threonine (5.0-6.5 mg/100 g), and low tyrosine (2.0-3.0 mg/100 g) levels relative to other dairy proteins. In some embodiments, a more hydrophobic protein fraction improves the stability of the suspension. Thus, in some embodiments, the protein comprises predominantly hydrophobic amino acid side chains, as determined by direct sequencing of amino acids, by the Wimley-White interface or octanol scales, by water micro-droplet contact angle measurements, and/or by other common measures of peptide and protein hydrophobicity.

In some embodiments, the protein comprises a whey protein, a casein protein, an egg white protein, an insect protein, a plant protein, or combinations thereof. In some embodiments, the plant protein is Maringa. In some embodiments, molecular weights for the protein, such as but not limited to whey proteins, vary from 18 kilodaltons (kD) to 25 kD, including 19 kD, 20 kD, 21 kD, 22 kD, 23 kD, 24 kD, and 25 kD. In some embodiments, the protein comprises at least about 20% of the caloric value of the composition, optionally at least about 21% of the caloric value of the composition, optionally at least about 22% of the caloric value of the composition, optionally at least about 23% of the caloric value of the composition, optionally at least about 24% of the caloric value of the composition, optionally at least about 25% of the caloric value of the composition.

In some embodiments, wherein the macronutrients are proteins and fats, the proteins and fats are present at ratio of between 1:1 and 1:3 protein to fat by weight, including any ratio combination with this range, e.g. 1:1.2, 1:1.5, 1:1.7, 1:2, 1:2.3, 1:2.6, 1:2.9, 1:3 and the like. In some embodiments, the fat predominates. In some embodiments, the protein is dispersed in fat at an approximately 1:1 ratio on a weight/weight basis, i.e., 50% protein/50% fat by weight.

In some embodiment, when the ingredient comprises a protein, the protein ingredient comprises has a pH ranging between 6.5 and 3.5, including pH 6.5, 6.0, 5.5, 5.0, 4.5, 4.0, and 3.5 when hydrated. The pH of the hydrated protein is indicative of the process used to produce it. By way of example and not limitation, the proteins used in the studies described herein below reflect the pH of normal bovine milk (˜6.5) or proteins at pH ranges below that of bovine milk. In some cases, lower pH could be a result of microbial fermentation related to cheese production or to direct addition of acid to lower pH. In some embodiments of a composition of the presently disclosed subject matter comprises a calcium level below 900 mg per 100 grams protein. Furthermore, in some embodiments when the ingredient is a protein, the sugar content of the protein source comprises less than about 2% by weight, such as below about 1.79% by weight. To elaborate, in some embodiments, low sugar (such as lactose) in a dairy protein would lessen browning reactions that could limit shelf life. Furthermore, in some embodiments when the ingredient is a protein, the water content of the protein source comprises less than about 6% by weight, such as below about 6, 5, 4, 3, or 2% by weight. By way of further exemplification and not limitation, a dry protein powder may more easily and fully mix with pure fat which, by definition, has a very low water content.

In some embodiments, the composition comprises a carbohydrate. In some embodiments, the carbohydrate comprises a sugar, a sugar syrup, a polyol, a flour, a starch, a fiber or hydrocolloid, or combinations thereof. In some embodiments, the carbohydrate comprises at least about 40% of the caloric value of the composition, optionally at least about 45% of the caloric value of the composition. In some embodiments, the carbohydrate is dispersed in fat at a ratio varying from 1:1 to 2:1 on a carbohydrate to fat weight/weight basis, e.g. 1:1, 1.1:1, 1.2:1, 1.3:1, 1.4:1, 1.5:1, 1.6:1, 1.7:1, 1.8:1, 1.9:1, 2:1, and the like.

In accordance with aspects of the presently disclosed subject matter, approaches for maintaining fine particle dispersions and keeping the particles suspended until the solid fat hardens are provided, as are approaches for accomplishing this on a large scale. Particle size of dispersed materials and heating/cooling of fat are aspects of these approaches.

In some embodiments of the presently disclosed compositions, fats are the primary dispersing agent. In some embodiments, the dispersion is facilitated by inclusion of proteins with a high degree of hydrophobicity and a fine particle size, as mentioned elsewhere herein. Additionally, mixing of proteins with other dry ingredients in a particular order can facilitate the maintenance of a uniform dispersion in the fat matrix. By way of non-limiting example, sugar, starches and proteins can be mixed together first in order to disperse micronutrients and other dry ingredients present at low concentrations. Alternatively, as another non-limiting example, protein can first be mixed with liquid or partly melted fat in order to encourage maximum hydrophobic interactions. The pH of the protein used may first be adjusted to a point close the pI (isoelectric point) in order to minimize charge and maximize hydrophobic interactions. The other dry ingredients would then be added in. In some embodiments, other ingredients (e.g., a natural plasticizer like glycerin) are employed.

In some embodiments, a composition in accordance with the presently disclosed subject matter comprises an ingredient, such as but not limited to a macronutrient, a micronutrient, a dietary bioactive, and/or a pharmaceutical ingredient, having a particle size is that does not exceed a size that can be perceived as unacceptable in texture or mouthfeel by human consumption. In some embodiments, the ingredient has a particle size that enables the material to be considered an acceptable direct-addition food ingredient. In some embodiments, the ingredient has a particle size ranging from about 10 microns to about 200 microns, including any value within this range, such as but not limited to 12 microns, 25 microns, 50 microns, 75 microns, 100 microns, 125 microns, 130 microns, 150 microns, 175 microns, 180 microns and 200 microns.

In some embodiments, the ingredient has a D90 value for particle size of about 100 microns or less. Particle size production techniques, such as but not limited to food-based particle size production techniques, do not necessarily produce uniformity of size, but rather maintains a typical bell-curve distribution. Thus, particle sizes are typically defined by D-values, where D-90 equates to 90% of particle sizes falling below stated value. D-50 and/or D-10 are often cited as reference points along with D-90. In some embodiments, the particle size criteria to enable the ingredient to be considered an acceptable direct-addition food ingredient comprise a D90 value for particle size of about 100 microns or less, in some embodiments about 95 microns or less, in some embodiments about 90 microns or less, in some embodiments about 85 microns or less, in some embodiments about 80 microns or less, and in some embodiments, about 75 microns or less. In some embodiments, the ingredient has a D50 value for particle size of about 50 microns or less, in some embodiments 25 microns or less, in some embodiments, 10 microns or less.

Representative particle size goals for ingredients in accordance with the presently disclosed subject matter include:

D-90=100 micron, 95 micron, 90 micron, 85 micron, 80 micron, or 75 micron.

D-50=50 micron, 25 micron, or 10 micron.

D-10=inconsequential.

Dietary bioactive applications and/or pharmaceutical applications (e.g., prebiotics, probiotics, antioxidants, compounding of various medications) can include blending active ingredients in a fat matrix to slow their release, especially for pharmaceuticals that “should be taken with food.” This could also extend to dietary bioactive products that are being created in the market in edibles (e.g. cannabis-based products). In some embodiments, an effective amount of a dietary bioactive ingredient and/or pharmaceutical ingredient is included in the composition.

In some embodiments, the composition comprises an ingredient selected from the group comprising a plasticizer, an emulsifier, a thickener, an antioxidant, a chelator, a flavoring, caffeine, theobromine, and combinations thereof.

Representative plasticizers include but are not limited to glycerin. Representative emulsifiers include, but are not limited to, lecithin and other phospholipids, mono and diglycerides, tweens and spans. Representative thickeners include but are not limited to gums (e.g., xanthan gum and guar gum), hydrocolloids, and syrups. Representative antioxidants include but are not limited to vitamin E and BHT. Representative chelators include, but are not limited to EDTA and citric acid. Use levels for the above ingredients can vary between 0.01% to 5%, depending on the ingredient in question.

Flavoring ingredients can also be included in the presently disclosed compositions. Desirable flavors include both sweet (e.g., chocolate) and savory (e.g. cheese) flavors, with and without caffeine. Additional optional characteristics include maintenance of flavor and nutrition, as well as a lack of bar hardening, melting, and discoloration over an approximately two-year shelf life. Representative flavorings are disclosed in the Examples, set forth herein below. Sweet and savory flavors are provided, such as but not limited to chocolate, cheese and crackers, cheddar bacon and jalapeno bacon. (Use levels would vary based on flavors from the 1-200 parts per million level for pure flavor active compounds to the 1-10% (by weight or by volume) level for food-based flavors, such as cheese powder.

The presently disclosed compositions can be used in accordance with a variety of applications as would apparent to one of ordinary skill in the art upon a review of the instant disclosure. Representative applications included but are not limited to shelf-life extension; delivery and/or protection of protein, vitamins, and other labile nutrients; flavor protection; probiotic delivery vehicle; and/or fiber delivery vehicle.

In some embodiments, the composition is provided in the form of a bar, a filling, a ganache, a pastry, a shortbread, an icing, a yogurt, a butter, a paste, a gel, a pellet, a sphere, or an ovoid disc. Thus, in addition to bars, provided are spherical or ovoid candies, which can be chocolate, candy or gelatin-coated, but do not have to be chocolate, candy or gelatin-coated. Such candies can comprise a similar protein/fat composition as a bar as disclosed herein, but are of smaller size than bars in order to allow for an alternate approach for delivering a very similar nutrient profile or foods that are bite size for quick delivery in activities like rock climbing when light, portable products are desired.

Representative applications thus include M&M-like or round/ovoid small serving size products with a similar nutritional profile as bar embodiments; highly fortified nut butters using more plastic or semi-solid fats; cheese fillings for crackers; sweet fillings for cookies; icing; ganache or any confections requiring high fat content; pastries; shortbreads; Ice cream inclusions; cheese products (cream cheese, processed cheese sauce product); thickened yogurts; modified butters; long-term nutrient storage without refrigeration; high protein “edibles” that contain CBD or other active ingredients in markets where their sale is permitted; and the dispersion of pharmaceuticals best consumed with food. The presently disclosed compositions thus shift protein bar, sports nutrition, pharmaceutical, and “edibles” design and could utilize modified chocolate/confection processing lines for, as well as design of high fat- or oil-based food products that people consume.

### Processes for Making Compositions

The presently disclosed subject matter provides processes to make the above-described compositions to uniformly disperse an ingredient (e.g. proteins/carbohydrates/etc.) in a fat matrix. In some embodiments, disclosed is a process for preparing a composition comprising a dispersion of an ingredient, such as but not limited to a macronutrient, micronutrient, dietary bioactive, and/or pharmaceutical ingredient, in a fat matrix. In some embodiments, the process comprises heating the fat matrix to provide a melted or heat-plasticized fat matrix; mixing the ingredient into the melted or heat-plasticized fat matrix; and cooling the mixture to form the composition comprising a dispersion of the ingredient, such as but not limited to a dispersion of a macronutrient, micronutrient, dietary bioactive, and/or pharmaceutical ingredient, in a fat matrix. Other ingredients can also be mixed into the fat matrix, including but not limited to a plasticizer, a thickener, an antioxidant, a chelator, a flavoring, and combinations thereof. In some embodiments, the dispersion is a uniform dispersion. The heating can be done to any desired temperature to melt the fat matrix, such as based on the fat matrix components chosen, as would be apparent to one of ordinary skill in the art upon a review of the instant disclosure. By way of example and not limitation, the heating can be done at about 50° C. In some embodiments, the heating and/or cooling comprises tempering the mixture; however, tempering is not required.

In some embodiments, the construction of the matrix begins with melted fat incubated for a predetermined period of time, such as about 1-3 minutes. In some embodiments, the melted fat is also held at a predetermined temperature, such as but not limited to about 25° C. By way of exemplification and not limitation, after the fat melts, it is held for one minute at 25° C. Including a hold time is an aspect of a process of the presently disclosed subject matter, as processes in the are typically designed to quickly move to bar ingredient mixing and bar molding steps before fats re-solidify. In some embodiments, once fat has been held for a desired hold time, such as one minute, all the dry ingredients are added and the product is mixed with a low shear rate, e.g., less than 100 rpm or a shear rate of less than 170 seconds⁻¹. Monitoring shear rate is also an aspect of some embodiments of the presently disclosed subject matter. That is, it can be the case that if too much shear is applied in the mixing process, the matrix does not form correctly. Thus, the method further comprises using a low shear mixing method, such as in either a vertical or horizontal mixer. The composition is then formed using a slab roller, such as a heated slab roller. In some embodiments, an extrusion method is not employed.

In some embodiments, the method comprises forming the composition into a desired shape or format, such as but not limited to a bar, a filling, a ganache, an icing, a yogurt, a butter, a paste, a gel, a pellet, a sphere (with or without a food-grade candy shell), or an ovoid disc (with or without a food-grade candy shell). In some embodiments, forming the composition comprises layering the composition into a mold or form of a desired shape. The method further comprises using a low shear mixing method, such as in either a vertical or horizontal mixer. The composition is then formed using a slab roller, such as a heated slab roller. In some embodiments, an extrusion method is not employed.

In some embodiments, the presently disclosed subject matter provides approaches to more homogenously mix fat and protein together and to examine the effects of proteins with differing degrees of hydrophobicity on bar prototypes.

The materials and equipment employed include basic industrial kitchen (hot plate, mixing bowls, sauce pot, scale, slab roller) and chocolate tempering machine for compositions that include chocolate. However, tempering is not required. In some embodiments, the presently disclosed processes employ modified chocolate/confection processing lines for manufacture. In the case of the preparation of spherical or ovoid candies, which can be candy coated, a form of the desired size is used and then the formed composition is placed in a standard tumbler for candy coating. In the case of compositions comprising chocolate, a conching step can be performed to enhance smoothness.

Obtaining and maintaining a fine dispersion of particles may be achieved in a number of ways, including but not limited to the use of various types of vertical and horizontal tumblers and mixers, with or without scraped surface. Rotation, vibration, ultra-sound, or electric current (in the case of charged particles) may be used to maintain a fine particle dispersion until the mixture solidifies, gels, freezes, or is otherwise stabilized.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

The examples include both chocolate and cheese flavors in caffeinated and non-caffeinated varieties. In one example a more savory bar composition was prepared. A cheese or “cheese and crackers” flavor was viewed as the best approach. Glycerin and a potato starch slurry were used to adjust texture and water activity in the cheese flavor.

TABLE 1
Chocolate Bar Prototype Nutrient Profile and Ingredients

| | | |
|---|---|---|
| Nutrient/Property | Value | Percent of Calories |

| Total Calories | 723.5 | | N/A |
|---|---|---|---|
| Protein | 34 | grams | 19% |
| Carbohydrates | 62.5 | grams | 35% |
| Fat | 37.5 | grams | 47% |
| Fiber | 1.2 | grams | N/A |
| Bar Weight | 134 | grams | N/A |
| Water Activity | 0.50 | | N/A |
| Melting Point | >40° | C. | N/A |

Ingredients (listed in order by weight): Cocoa liquor, Whey protein Isolate, Oat Flour, Invert sugar, Cocoa Butter, Palm Kernel Oil, Cocoa Powder, Salt, Caffeine.

TABLE 2
Savory Bar Prototype Nutrient Profile and Ingredients

| | | |
|---|---|---|
| Nutrient/Property | Value | Percent of Calories |

| Total Calories | 710 | | N/A |
|---|---|---|---|
| Protein | 35 | grams | 20% |
| Carbohydrates | 66 | grams | 37% |

| | | |
|---|---|---|
| Fat | 34 grams (zero trans) | 43% |

| | | | |
|---|---|---|---|
| Fiber | 1.2 | grams | N/A |

| | | |
|---|---|---|
| Bar Weight | 140 grams/4.9 ounces | N/A |
| Water Activity | 0.65 | N/A |

| | | | |
|---|---|---|---|
| Melting Point | >40° | C. | N/A |

Ingredients (listed in order by weight): Whey protein Isolate, Oat Flour, Cheddar Cheese Powder, Invert Sugar, Cocoa Powder, Palm Kernel Oil, Potato Starch, White Cheese Powder, Corn Syrup, Glycerin.

### Use of Reducing Sugars in Cheese Bars

The use of reducing sugars, especially in cheese bars, caused non-enzymatic browning reactions to occur when exposed to high temperatures (40 degrees Celsius) or to weeks of storage at room temperature. This might be partly due to packaging.

### Model Protein Bar System

Fat/protein combinations and processes were tested both in a nutrient-dense protein bar model system and in an isolated fat/protein model. Referring to the protein bar model first, the reason for testing fat/protein combinations in a bar model was to provide an extended shelf-life protein bar for the military. Currently in the protein bar market, protein bars fall into two general categories: extruded bars, with or without layers, and multi-component bars held together with sugar syrups. Extruded bar layers may or may not contain inclusions and the entire bar is often enrobed in a chocolate-like coating. A popular example of the extruded, enrobed bar approach would be PowerBar® products. The extruded bar coatings melt at around 34° C. and the extruded center hardens over six months to a year of storage. Examples of the “glued together” approach would include Clif Bars® and Friend Bars®. These are mixtures of ingredients “glued” together with a sugar syrup. These “glued” type bars typically contain significant quantities of unsaturated fat, resulting in short shelf lives due to rancidity.

In contrast, the presently disclosed bars use a solid fat matrix to suspend homogenously dispersed particles, in a way that is analogous to chocolate (in terms of suspending solid particles in fat). Although there is an analogy to chocolate, the model bars are quite distinct from chocolate in terms of composition and processing, such as but not limited to with regard to their high protein content, relatively low sugar content, fat combinations, and lack of tempering. The resulting model protein bar has higher melting temperature and longer shelf life than bars currently on the market, due to particular ingredient mixtures and processing.

In some embodiments, the ingredient matrix disclosed herein encapsulates protein within solid fat, allowing the two macronutrient components to become bound together in a way that prevents separation. In a particular, non-limiting example, the model bar-matrix uses three key ingredients: whey protein isolate, cocoa butter, and palm kernel oil. Choosing a mixture of cocoa butter and palm kernel oil (PKO) was not originally planned as these fats do not generally work well together. In some embodiments, pure cocoa butter is used due to its shelf stability and high melting temperature. But, after finding that even higher melting temperatures were needed, mixtures of cocoa butter and PKO were tested. When cocoa butter and PKO are mixed, they typically “bloom”, meaning that the mixed fats produce large crystals that can move to the surface of the food, disrupting appearance and texture. Cocoa butter and cocoa products' ideal crystalline structure, termed “beta”, has a melting point of 34-35° C. and is stable until that point. (Minifie 1999). Conversely, PKO assimilates into beta-prime crystals. When PKO is mixed with cocoa butter products, it typically becomes the dominating crystal structure and seeds the cocoa butter with a less desirable (grainy) beta prime crystalline structure. This is why chocolate companies do not mix PKO with cocoa butter for the production of chocolate bars. Pure PKO can, however, be used as a cocoa butter replacement also known as Cocoa Butter Equivalent (CBE). Thus, an aspect of the presently disclosed subject matter is the successful combination of cocoa butter and PKO without fat bloom in a bar system.

In an exemplary bar model system, six core ingredients were employed: whey protein, cocoa liquor (a natural mixture of 50% cocoa solids and 50% cocoa butter), sucrose syrup (a mixture of water and sucrose), oat flour, cocoa powder, and salt. These ingredients were combined to create a slab-rolled bar product. The process of making the model system includes the solid fats being melted at or above 50° C. After the fat melts, it is held for one minute at 25° C. Including a hold time is an aspect of a process of the presently disclosed subject matter, as processes in the are typically designed to quickly move to bar ingredient mixing and bar molding steps before fats re-solidify. Once fat has been held for a desired hold time, such as one minute, all the dry ingredients are added and the product is mixed using a pastel style mixing process with a low shear rate, e.g., less than 100 rpm or a shear rate of less than 170 seconds⁻¹. Monitoring shear rate is also an aspect of some embodiments of the presently disclosed subject matter. That is, it can be the case that if too much shear is applied in the mixing process, the matrix does not form correctly. This style of mixing is also in contrast to typical processes in the art, which aim for a fast, high rate of mixing to create the product quickly. A horizontal mixer can also be used at low speed to allow the product to mix in relatively low shear rate conditions. When mixing of the product is complete, the matrix is clay-like and malleable enough to form any shape.

The next step is to load the mixture into a hopper for metering onto a slab-rolling surface. This is in contrast to an extrusion process. It was observed that if the product is extruded while warm and malleable, the pressure of extrusion forces the bonding between the protein (e.g., whey protein) and the fat to collapse, causing the product to lose the fat from the matrix. If extrusion continues, the loss of fat from the matrix will eventually cause mechanical failure of the extruder as the product cools. This result is surprising, as most many bar producers use extruders instead of rolling.

The model bars of the presently disclosed subject matter have been subjected to extensive shelf life testing, both accelerated testing (one year at 55° C.) and two years at 25° C. without significant declines in sensory quality.

Over the course of model bar testing, PKO was introduced to the original set of six ingredients and a variety of flavors were produced (peanut butter, cheese, caramel, etc.) During model bar testing it became apparent that the most stable fat to protein ratio was 50/50 weight/weight, regardless of changes made to other ingredients. The mixture of 50/50 fat/protein can be adopted to a large number of products. Any product that can be produced using a high saturated fat mixture such as ganaches, pastries, shortbreads, or other food systems. The presently disclosed subject matter can also be used to introduce a significant source of protein into these and other food systems. By changing to other fat sources for the formation of the matrix of fat and protein or substituting part of the fat component, one can achieve a higher protein product without a dramatic change in flavor or texture. For example, a croissant might be constructed substituting one-third of the butter with the matrix of 50% cocoa butter and 50% whey protein. The newly constructed croissant-like pastry will contain whey protein that can be used in place of adding eggs or meat-based products for a breakfast sandwich. Another example might be to take the ganache from inside of a chocolate truffle and replace some of the cocoa liquor with this matrix adding more protein while not disturbing the texture or flavor. Thus, the presently disclosed subject matter provides for unexpected new products, as it is believed that one of ordinary skill in the art would not think about replacing saturated fat with a fat/protein matrix to create a healthy high protein product.

### Analyses of Dairy Proteins Used in Model Systems

The observation that cocoa butter and PKO combinations, mixed with 50/50 weight/weight ratio, produced highly stable model protein bars led to closer analysis of the specific types of fat and protein that could be used to optimize stability. While cocoa butter and PKO are relatively homogenous ingredients with known compositions, the dairy protein ingredients used were much more variable. The protein powders used were (all commercially available from Glanbia plc, Kilkenney, Ireland): Barpro 585, a milk protein isolate (contains both casein and whey proteins), Barpro 288, a dairy protein blend (contains both casein and whey proteins), Avonlac 180, a concentrated whey protein, Provon 190, a whey protein isolate, and Bevwise A-100W, a whey protein isolate. In order to better characterize the fat/protein matrix, moisture and particle size analyses were conducted on the protein powders used. Moisture was measured using the Official Methods of Analysis, (AOAC International) Method 927.05. and is reported in Table 3, along with percent protein, sugar, and calcium. Protein particle size distribution was determined using a Microtrac Sympatec (Microtrac Inc., Montgomeryville, Pa., United States of America) particle size analyzer and a Gilson (Gilson Co., Inc., Lewis Center, Ohio, United States of America) sieve shaker, Model SS-15 as per AOAC 973.03. Protein particle size ranged from 12 μm and 180 μm, with the bulk in the range of 50 um to 120 um. Particle size varied between the isolate type. For Barpro 288, the range is described as having a mean volume (MV) of 70.926 μm with a standard deviation (SD) of 48.67 μm. When looking at Avonlac 180, it has an MV of 21.416 μm and SD of 26.99 μm. Next, Barpro 585 has a MV of 63.18 μm with SD of 34.43 μm. Proven 190 has an MV of 48.37 μm with an SD of 25.54 μm.

TABLE 3
Moisture, Protein, Sugar and Calcium Contents in Proteins Tested

| Protein | % Moisture | % Protein | % Sugar | mg Calcium * |
|---|---|---|---|---|
| Barpro 288 | 4.14 | 90.36 | 1.79 | 945.20 |
| Barpro 585 | 5.29 | 86.14 | 4.0 | 2,100.00 |
| Avonlac 180 | 4.00 | 80.79 | 1.7 | 880.00 |
| Provon 190 | 3.61 | 94.02 | 2.0 | 402.00 |
| BevWise A100W | 2.36 | 84.10 | 0.7 | 480.00 |

*mg calcium reported on a per 100 gram whey protein basis.

Whey protein compositions were analyzed via acid-hydrolysis to liberate amino acids, followed by UPLC analysis, as per AOAC method 982.30. Cystine and methionine were calculated using AOAC Method 982.30. Tryptophan analysis was conducted using AOAC Method 988.15. Amino acid profiles among proteins tested were similar, differing primarily in the relative amounts of aspartic acid, threonine, and tyrosine, as shown in Table 4. Lowest values are in bold.

TABLE 4
Amino Acid Content Of Various in Proteins Tested

| | Aspartic Acid | Threonine | Tyrosine |
|---|---|---|---|
| Protein Powders | mg/100 g | mg/100 g | mg/100 g |
| Barpro 288 | 9.1 | 5.6 | 3.5 |
| Barpro 585 | 5.9 | 3.3 | 4.3 |
| Avonlac 180 | 8.7 | 5.3 | 2.2 |
| Provon 190 | 9.9 | 6.1 | 2.6 |
| BevWise A100W | 8.8 | 5.9 | 2.4 |

### Isolated Fat/Protein Mixtures

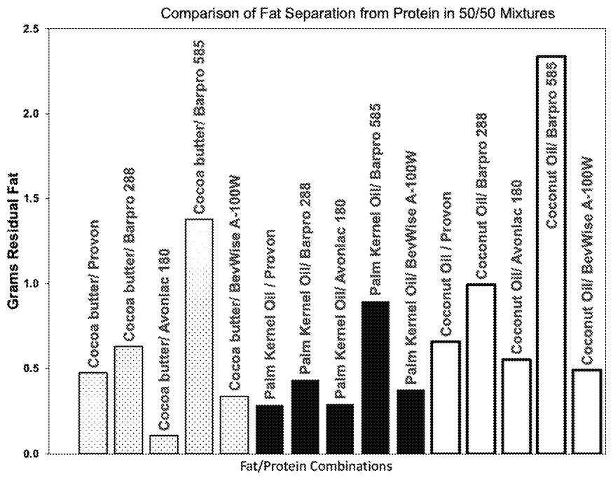

A rapid method was developed to determine the stability of specific fat/protein ratios outside of a food matrix. Briefly, 10 grams of solid fat was melted, mixed with varying weights of dairy proteins (0.0, 2.5, 5.0, 7.0, and 10 grams). The mixtures were re-solidified to create weight/weight fat/protein ratios of 100/0, 80/20, 70/30, 60/40, and 50/50. These fat/protein mixtures were placed onto a filter in the upper portion of 50 ml centrifugation tubes. The fat/protein mixtures were heated to 3° C. above the melting point of the fat and centrifuged in order to determine how much fat would separate from each fat/protein mixture. The best fat/protein ratio in terms of fat retention was found to be 50/50, which reflects what was observed in the bar model. FIG. 1 shows the differences in fat loss for varying fat/protein combinations at weight/weight ratio of 50/50. Three proteins, all proteins comprising whey, provided the highest degree of stability (least fat loss) across all fats tested: Provon 190, Avonlac 180, BevWise A-100w.

### Discussion of Examples

Commercially available protein powders have a wide range of functional properties and can be used in a variety of foods and beverages. Due to the dynamic nature of protein interactions within food matrices, product developers must be careful in the selection of protein powder based on the desired interactions needed in their system. For the presently disclosed subject matter, it was desired to elucidate the properties needed in a protein for the creation of a stable fat/protein matrix. We determined that a desirable particle size for fat/protein interactions is between 11 μm and 130 μm, based on the superior performance of the three whey proteins. These factors place Avalac 180, Provon 190, and Bevwise A-100w, inside the range of protein with particle sizes needed to develop ideal mixtures. Barpro 288 and 585 had mean volumes of 70 μm and 63 μm respectively at the upper end of the desired particle size range. However, considering the variability around the mean volume, SD of 48.67 μm and 34.43 μm respectively indicates that these protein powders tend to have particles that range higher than that of the desired protein systems. While it is not desired to be bound by any particular theory of operation and considering the underlying cause for the ideal particle size range, it is postulated that smaller particles should be absorbed into the saturated fats more efficiently allowing for better binding and a potentially smoother product. It was also observed that a mixture comprising greater than 60% protein to 40% fat were very grainy and powdery. While it is not desired to be bound by any particular theory of operation, this larger amount of protein tends to encourage protein to protein interactions, resulting in clumping of whey protein within the fat mixture. The protein and fat matrix can be adjusted to meet the requirements for many different products, not just protein bars.

The moisture content of the protein (e.g., whey protein) is desirably at a low level for our fat containing matrix. While it is not desired to be bound by any particular theory of operation, it is believed that the low moisture protein powders are better able to associate with the lipophilic and hydrophobic fats. The data provided herein supports a connection between the moisture content of the whey protein powder and its solubility into fats. With this idea in mind, it is desired in some embodiments that the moisture content for a stable matrix needs to be below 4%. While it is not desired to be bound by any particular theory of operation, it is believed that the low moisture ensures that the hydrophobicity of the powder is not repressed and allows for the absorption of protein into the melted lipid mixture. Both of the Barpro products have a moisture content above 4% (Barpro 288 at 4.14% and Barpro 585 at 5.25). Thus, the analysis presented herein predicts that these higher moisture values will inhibit stable matrix formation. The presently disclosed novel analytical method proved useful for testing the ability of various commercially available proteins and fats to form a stable matrix. Of the five protein powders tested, the whey protein isolates performed better in forming a stable matrix. While the other powders contained some whey, they also contained other proteins. In addition to the difference in protein composition between whey protein isolate (WPI) and the other powders there are significant processing differences in the production of these powders. The process of producing an isolate is vastly different from making a concentrate or dairy blend. For example, the WPI is a result of cheese making while the concentrate is made from separating the whey from the milk in an ultrafiltration step. As for the milk protein isolate, the isolation process seeks to partially remove non-protein components, from low-fat milk with the aim of producing a mixture of casein and whey protein product. The difference between the three different processes briefly described above shows that the methods produce a different complement of proteins should be considered in selecting a protein for a protein fat matrix.

Furthermore, the sugar content of the sampled proteins varies significantly from 0.7 to 4.0 grams of sugar per 100 grams protein powder. This is an extensive range, and adds to the noted differences between the proteins produced by different separation or recovery processes. A trend was observed in the association of protein to fat such that when the sugar content for a protein powder was below 1.7% sugar the ability to bind with desirable fats increases. Those powders with sugar content below 1.7% are Avonlac 180, Provon 190, and BevWise A-100w. The test samples with sugar contents above 1.7% were the two Barpro products. Thus, sugar content can also be considered in preparing a protein fat matrix.

The calcium content of the protein powders also correlates with the overall ability of the protein to bind to the fat matrices. Higher calcium contents are correlated with less than desired matrix formation. Levels of calcium lower than 900 mg of calcium per 100 grams of protein are found in powders possessing the ability to form stable protein fat interactions. The products that fall into the ideal range include: Avonlac 180, Provon 190, and BevWise A-100W. The two Barpro powders have levels of calcium above the desired range. In fact, the Barpro 585 has 2.1 grams of calcium per 100 grams powder. It was observed that Barpro 585 formed the least stable matrices with all fat types. While it is not desired by any particular theory of operation, it helps support the connection that high calcium amount might repel fat:protein interactions by making the proteins more hydrophilic. Thus, calcium content can also be considered in preparing a protein fat matrix.

Avonlac 180, Provon 190, and BevWise A-100W, differed from the other proteins tested with regard to their contents of three amino acids: aspartic acid, threonine, and tyrosine. Both aspartic acid's R-group and threonine's R-groups are considered polar. Tyrosine is the only amino acid of the three with a hydrophobic R-group. Thus, amino acid side chains and overall protein hydrophobicity can also be considered in preparing a protein fat matrix.

When looking at the lipid-protein matrix, the connections between the types of fat and the protein types determines how much residual fat leaves the matrix. Cocoa butter and palm kernel oil appear to have low residual fat particularly when tested with the Provon, Avolac, and BevWise proteins. All of the Barpro 585 seems to have low fat retention for those that are higher in saturated fats and seems to be very high with canola oil. Both of the Barpros seems to work opposite to the other three types of proteins. Cocoa butter and palm kernel oil are cited as two fats that are not recommended for tandem use in a food system because cocoa butter forms beta while PKO forms beta-prime crystalline structures. While coconut oil's most common binding crystallization forms are reported as beta-prime and alpha. (Ribeiro and others 2015) While it is not desired to be bound by any particular theory of operation, it might be the reason books and chocolate producers suggest that if other lipids are mixed with chocolate, the added fast must be less than 5% of the total fats. However, within the formation of representative fat and protein matrices of the presently disclosed subject matter, a mixture of PKO and cocoa butter closer to 10 percent PKO as shown to be desirable.

Based on the evidence presented, it is shown that the presently disclosed subject matter for the production of stable fat/protein matrices is practically useful and effective across a range of food systems. In some embodiments, the presently disclosed subject mixture comprises a stable protein in fat matrix comprising highly saturated lipids mixed with whey protein isolate. In one aspect, it was observed that substitution of whey concentrate with ultra-filtration results in less desirable protein-fat interaction. In a particular representative embodiment, the blend of fat and protein ratio was 50/50, using a mixture of cocoa butter and palm kernel oil. Using these saturated fats together in this matrix does not cause the fat bloom that typically occur with fat mixtures having dissimilar crystalline structures. In other particular embodiments, the moisture content of the WPI was observed to be below 5.0%. Other representative parameters for the composition include controlling pH between 6.5 and 3.5, calcium levels below 900 mg per 100 grams protein, relatively low sugar levels, high aspartic acid and threonine levels, and low tyrosine levels. Finally, a protein particle size between 10 μm and 130 μm was observed to be desirable in some embodiments. In some embodiments, the construction of the matrix begins with melted fat incubated for 1-3 minutes before using a low shear mixing method in either a vertical or horizontal mixer followed by using a heated slab roller. Thus, aspects of the presently disclosed subject matter involve fat/protein ratios, such as 50/50 fat/protein ratios; particular combinations of specific fats and protein, such as whey proteins; particular amino acid compositions in the proteins used; particular protein particle size ranges; the general desirability of whey proteins over mixed casein and whey; the ability of cocoa and palm kernel fats to work together effectively in this system; and the mixing and molding methods used in the bar model.

### REFERENCES

All references listed below, as well as all references cited in the instant disclosure, including but not limited to all patents, patent applications and publications thereof, and scientific journal articles are incorporated herein by reference in their entireties to the extent that they supplement, explain, provide a background for, or teach methodology, techniques, and/or compositions employed herein.
U.S. Pat. No. 6,346,284
Minifie B W. 1999. Chocolate, Cocoa, And Confectionery: Science and Technology. Gaithersburg, Md.: Aspen Publishers, 1999.
Ribeiro A P B, Masuchi M H, Miyasaki E K, Domingues M A F, Stroppa V L Z, de Oliveira G M, Kieckbusch T G. 2015. Crystallization Modifiers in Lipid Systems. Journal of Food Science and Technology 52(7): 3925-46.
Berk Z., Chapter 7, Mixing, In Food Process Engineering. 2013. Food process engineering and technology. Amsterdam, Academic Press, 2013

It will be understood that various details of the presently disclosed subject matter may be changed without departing from the scope of the presently disclosed subject matter. Furthermore, the foregoing description is for the purpose of illustration only, and not for the purpose of limitation.

1. A composition comprising a dispersion of an ingredient in a fat matrix, optionally wherein the ingredient is selected from the group consisting of a macronutrient, a micronutrient, a dietary bioactive, a pharmaceutical ingredient, and combinations thereof, further optionally, wherein the dispersion is a uniform dispersion.

2. The composition of claim 1, wherein the macronutrient is a protein, a carbohydrate, a second type of fat in addition to the one forming the primary fat matrix, water, or a combination thereof.

3. The composition of claim 1, wherein the fat matrix comprises a fat that is a solid at temperatures ranging up to 45° C.

4. The composition of claim 1, wherein the fat matrix comprises a fat that is semi-solid at temperatures ranging up to 40° C.

5. The composition of claim 1, wherein the macronutrients are proteins and fats and the proteins and fats are present at ratio of between 1:1 and 1:3 protein to fat by weight.

6. The composition of claim 1, wherein the macronutrient is a protein of about 18 to 25 kiloDaltons.

7. The composition of claim 1, wherein the ingredient comprises particles and the ingredient particles have a particle size ranging from about 10 microns to about 200 microns.

8. The composition of claim 1, wherein the ingredient has a D90 value for particle size of about 100 microns or less.

9. The composition of claim 1, having a caloric value ranging from about 4 kilocalories per gram to about 8 kilocalories per gram.

10. The composition of claim 2, wherein the protein comprises predominantly hydrophobic amino acid side chains, as determined by a technique selected from the group consisting of direct sequencing of amino acids, Wimley-White interface or octanol scales, water micro-droplet contact angle measurements, other common measures of peptide and protein hydrophobicity, or any combination of the foregoing.

11. The composition of claim 2, wherein the protein comprises a whey protein, a casein protein, an egg white protein, an insect protein, a plant protein, or combinations thereof.

12. The composition of claim 2, wherein the protein comprises at least about 20% of the caloric value of the composition, optionally at least about 25% of the caloric value of the composition.

13. The composition of claim 2, wherein the carbohydrate comprises a sugar, a sugar syrup, a polyol, a flour, a starch, a fiber or hydrocolloid, or combinations thereof.

14. The composition of claim 1, further comprising an ingredient selected from the group consisting of a plasticizer, a thickener, an antioxidant, a chelator, a flavoring, and combinations thereof.

15. The composition of claim 1, wherein the fat is a fat selected from the group consisting of cocoa butter, coconut oil, palm kernel oil, tristearin, and fractions and combinations thereof.

16. The composition of claim 1, having an average water activity below 0.85, optionally below 0.75.

17. The composition of claim 1, wherein the composition is provided in the form of a bar, a filling, a ganache, a pastry, a shortbread, an icing, a yogurt, a butter, a gel, a paste, a pellet, a sphere (with or without a food-grade candy shell), or an ovoid disc (with or without a food-grade chocolate, candy or gelatin shell/encapsulation).

18. A process for preparing a dispersion of an ingredient in a fat matrix, optionally wherein the ingredient is selected from the group consisting of a macronutrient, a micronutrient, a dietary bioactive, a pharmaceutical ingredient, and combinations thereof, further optionally, wherein the dispersion is a uniform dispersion, the process comprising:
(a) heating the fat matrix to provide a melted or heat-plasticized fat matrix;
(b) mixing the ingredient into the melted or heat-plasticized fat matrix, optionally wherein the ingredient comprises particles; and
(c) cooling the mixture formed in step (b) to form the composition comprising a dispersion of an ingredient in a fat matrix, optionally wherein the ingredient is selected from the group consisting of a macronutrient, a micronutrient, a dietary bioactive, a pharmaceutical ingredient, and combinations thereof, further optionally, wherein the dispersion is a uniform dispersion.

19. The process of claim 18, wherein the fat matrix comprises a fat that is a solid at a temperature ranging up to 45° C.

20. The process of claim 18, wherein the fat matrix comprises a fat that is semi-solid at temperatures ranging up to 40° C.

21. The process of claim 18, wherein the fat is a fat selected from the group consisting of cocoa butter, coconut oil, palm kernel oil, tristearin, and fractions and combinations thereof.

22. The process of claim 18, wherein the macronutrient is a protein, a carbohydrate, a fat, water, or a combination thereof.

23. The process of claim 18, wherein the macronutrients are proteins and fats and the proteins and fats are present at ratio of between 1:1 and 1:3 protein to fat by weight.

24. The process of claim 18, wherein the macronutrient is a protein of about 18 to 25 kiloDaltons.

25. The process of claim 18, wherein the ingredient has a particle size ranging from about 10 microns to about 200 microns.

26. The process of claim 18, wherein the ingredient has a D90 value for particle size of about 100 microns or less.

27. The process of claim 18, having a caloric value ranging from about 4 kilocalories per gram to about 8 kilocalories per gram.

28. The process of claim 22, wherein the protein comprises predominantly hydrophobic amino acid side chains, as determined by a technique selected from the group consisting of direct sequencing of amino acids, Wimley-White interface or octanol scales, water micro-droplet contact angle measurements, other common measures of peptide and protein hydrophobicity, or any combination of the foregoing.

29. The process of claim 22, wherein the protein comprises a whey protein, a casein protein, an egg white protein, an insect protein, a plant protein, or combinations thereof.

30. The process of claim 22, wherein the protein comprises at least about 20% of the caloric value of the composition, optionally at least about 25% of the caloric value of the composition.

31. The process of claim 22, wherein the carbohydrate comprises a sugar, a sugar syrup, a polyol, a flour, a starch, a fiber or hydrocolloid, or combinations thereof.

32. The process of claim 18, further comprising an ingredient selected from the group consisting of a plasticizer, a thickener, an antioxidant, a chelator, a flavoring, and combinations thereof.

33. The process of claim 18, having an average water activity below 0.85, optionally below 0.75.

34. The process of claim 18, comprising holding the melted or heat-plasticized fat matrix for a period of time prior to the mixing.

35. The process of claim 34, wherein the holding of the fat matrix is carried out for 1 to 3 minutes.

36. The process of claim 18, mixing the ingredient into the melted or heat-plasticized fat matrix using a low shear mixing method.

37. The process of claim 18, comprising forming the composition into a bar, a filling, a ganache, a pastry, a shortbread, an icing, a yogurt, a butter, a paste, a gel, a pellet, a sphere (with or without a food-grade chocolate, candy or gelatin shell/encapsulation), or an ovoid disc (with or without a food-grade chocolate, candy or gelatin shell/encapsulation).

38. The process of claim 37, wherein forming the composition comprises forming the composition on a slab roller.
